(19) United States
(12) Patent Application Publication
(10) Pub. No.: US 2002/0137901 A1
(43) Pub. Date: Sep. 26, 2002

(54) Synthesis, and photodynamic therapy-mediated anti-cancer, and other uses of chlorin e6-transferrin

(76) Inventor: Philip Gerard Cavanaugh, Redford, MI (US)

Correspondence Address: Philip G. Cavanaugh, 3 StarThistle, Irvine, CA, 92604, US

(21) Appl. No.: 10/046,386

(22) Filed: Jan. 16, 2002

Related U.S. Application Data
(60) Provisional application No. 60/262,674, filed on Jan. 22, 2001.

(51) Int. Cl. C07K 14/79

(52) U.S. Cl. 530/400

(57) ABSTRACT

The invention describes the synthesis and proposed usage of a tumor-specific, site-specific tumor cell-killing agent. The agent binds to tumor cells with high affinity and at the same time will bind minimally to surrounding normal cells. The agent has conjugated to it a porphyrin, which when exposed to light, generates cell-killing reactive oxygen species. Thus, in areas which can be irradiated by light, a site-specific, tumor-specific cell killing can occur. The agent consists of the iron-transport protein transferrin (Tf) which is conjugated with the porphyrin chorin e6 (Ce6). For this patent, a novel method of conjugation was developed as conventional methods of conjugation of chlorin e6 to the protein resulted in the loss of transferrin's biological activity. The new conjugation procedure results in the covalent attachment of chlorin e6 to transferrin and yet maintains the natural activity of the protein. The synthesis occurs while the protein is immobilized to QAE-sephadex, in the presence of the zwitterionic detergent CHAPS (3-[(3-cholidamidopropyl) dimethylammonio]-1-propanesulfonate). Using this technique, the biological activity of the conjugated transferrin is preserved, the conjugate binds to cell surface transferrin receptors and promotes the growth of cells in culture, all while carrying the cell-killing chlorin e6. The conjugate induces a light-exposure dependent killing of tumor cells in tissue culture. After injection into cancer patients, a tumor cell killing effect will hypothetically be achieved by irradiation of the tumor site with light. The patent covers the new-found synthesis technique for and the in vitro and in vivo tumor cell killing usage of chlorin e6-transferrin.

### CROSS REFERENCE TO RELATED APPLICATIONS

None

### STATEMENT REGARDING FEDERALLY SPONSORED RESEARCH OR DEVELOPMENT

This invention was not directly supported by any federally sponsored research.

### REFERENCE TO SEQUENCE LISTING, TABLES, OR COMPUTER PROGRAM LISTINGS

None

### BACKGROUND OF THE INVENTION

Rapidly growing cells require continuous intracellular iron transport in order to divide. Free iron, or iron salts, are absent in biological systems as iron salts can catalyze many un-favorable reactions (Conrad and Umbreit, 2000). Therefore, all iron delivery, storage, and transport in cells and higher organisms occurs while the iron is complexed to proteins. The major circulating iron transport protein is transferrin (Tf), which exists in blood at levels of 200-400 mg/100 ml (Ponka and Richardson, 1998). Each transferrin protein binds and transports two atoms of iron. To accomplish iron internalization, cells express transferrin receptors (TfR; Testa, et. al., 1993; Ponka, et. al., 1998; Ponka and Lok, 1999) on their surface. These receptors interact with transferrin and two iron-saturated transferrins bind to one TfR. This TfR-Tf complex is internalized into the cell and the complexed iron is delivered to needed sites. Most tumor cells exhibit rapid growth rates and therefore internalize copious quantities of iron and express high levels of transferrin receptors (Gatter et. al., 1983; Niitsu et. al., 1987). Quiescent normal adult cells express little or no TfR (Gatter et. al., 1983; Tani et. al., 2000; Juhlin, 1989; Niitsu et. al., 1987). Therefore, in many tissue areas, if a tumor exists, the only site of high TfR expression will be associated with the tumor cells. The expression of TfR in human tumor cells has been found to correlate with tumor grade, stage, progression, and metastasis. This has been seen in breast carcinomas (Wrba et. al., 1986), bladder transitional cell carcinomas (Seymour, et. al., 1987), and malignant melanoma (Van Muijen et. al., 1990). In addition, high levels of TfR have been observed in a metastatic lesion of a maxillary neoplasm, but not in the parental tumor (Yoda et. al., 1994),and the expression of TfR was higher in a human melanoma line selected for metastatic capability in nude mice than in the poorly metastatic tumor cells of the parental population (Van Muijen, et. al., 1991). In other studies, growth response to Tf was seen to correlate with metastatic progression in the B16 melanoma (Stackpole et. al., 1994) and Tf was identified as the major bone-marrow derived mitogen for bone-marrow metastasizing prostatic carcinoma cells (Rossi et. al., 1992). We have found that tumor cell expression of TfR can correlate with the metastatic ability of certain tumor cells (Cavanaugh and Nicolson, 1991, 1998; Cavanaugh et. al., 1999), which indicates that heightened TfR expression can be associated with the more aggressive tumor cell types.

Therapy against cancer is ideal when cancer cells are specifically killed while normal cells are left largely intact. Furthermore, an ideal treatment is achieved when cell killing occurs only at the site of the tumor and any non-specific killing at other sites is avoided entirely. To achieve these ends, researchers designing anti-cancer therapies will direct cancer cell killing agents at cell components which are novel to cancer cells or are present at much greater numbers on cancer cells than on normal cells. Various toxin-conjugated or radioactive antibodies directed towards antigens expressed only on the surface of cancer cells have been produced and tested (Hudson, 1999; Scott and Welt, 1997). Strategies to combat cancer using reagents directed at the transferrin/TfR system are currently being explored, and these are most successful when used to treat tumors of hematopoetic origin (Elliot et. al., 1988; Kemp et. al., 1992, 1995; Kovar et. al., 1995). The problem with any agent of this nature is that they can act, albeit to a lesser degree, on normal cells nearby and distant from the tumor site, causing side effects. To circumvent the latter problem, treatments have been devised which attack cancer only at the site of the tumor. If a pre-toxin could be specifically delivered to the TfR, and could furthermore be specifically activated to the toxin state at a certain site, then a tumor cell specific, site specific killing of tumor cells could be achieved. If at the same time, the pre-toxin remained non-toxic at other sites where the activation was not performed, then side effects could be avoided.

Photodynamic therapy (PDT) is an anti-cancer strategy that has been the subject of intensive study in recent years (Hsi et. al., 1999). The idea is to deliver to a tumor site a an inactive toxin which is then activated to a cell-killing toxin by exposure to light. Site-specific light irradiation causes site-specific cell killing. A number of different compounds which become toxic when impinged upon by light have been developed (Hsi et. al., 1999). These compounds have been conjugated to various proteins (Akhlynina et. al., 1995; Donald et. al., 1991; Gijsens and De Witte, 2000; Del Govematore et. al., 2000 ) or covalently linked to other molecules (Katsumi et.al., 1994; Bachor et. al., 1991 ), to create a complex that when delivered in vivo, will produce a tumoricidal effect, when the tumor area is irradiated with light. One of the more useful PDT agents is chlorin e6, a nettle-derived porphyrin which is rendered toxic by irradiation with visible light.

We sought to conjugate transferrin with chlorin e6, to develop an anti-cancer PDT agent which would exploit the high affinity of tumor cells for transferrin and the site-specific nature of PDT. Transferrin has been suggested as a delivery vehicle for anticancer drugs (Singh, 1999) and non-chlorin e6 PDT conjugates of transferrin have been produced (Hamblin and Newman, 1994). However, follow-up studies and extensive in vitro or in vivo work with the latter have been lacking.

The conjugation of chorin e6 to proteins usually occurs in solution with compounds such as EDC (1-Ethyl-3-[3-dimethylaminopropyl] carbodiimide hydrochloride ) or cyclohexyl-3(2-morpholinoethyl) carbodiimide being present to activate chlorin e6 carbonyl groups to amine-reactive entities (Akhlynina et. al., 1995; Bachor et. al., 1991). With EDC, chorin e6 carboxyl groups form 0-acylisourea intermediates for their conjugation to protein primary amines. Typically, once reactions are complete, conjugated proteins are separated from un-reacted intermediate and chlorin e6 by gel filtration. A number of these procedures were used to conjugate chorin e6 to transferrin with apparent success at conjugate formation, however the conjugate made using these methods consistently displayed none of transferrin's usual growth stimulating activity on a particular target cell line. When conjugation using EDC was performed after immobilization of Tf to QAE-sepharose, biological activity of the ligand was maintained. The conjugated protein could be released from the gel by high salt only if a detergent such as CHAPS was present. Tf conjugated with chorin e6 in this fashion displayed cell growth-promoting activity, TfR binding activity, and displayed potent light-dependent killing of tumor cells in culture. As such, this patent and the invention is for this novel method for the conjugation of proteins to chorin e6, and for the subsequent use of this conjugate as a tumor-specific, tumor site-activatable, anti-cancer agent.

### BACKGROUND REFERENCES

U.S. Pat. Documents:

| | | |
|---|---|---|
| 5,528,453 | November, 1993 | Koopecek, et. al. |
| 5,876,989 | March, 1999 | Berg, et. al. |
| 5,976,535 | November, 1999 | Fritzberg, et. al. |
| 6,107,466 | August, 2000 | Hasan, et. at. |

Specific Documents:

Akhlynina, T. V., Rosenkranz, A. A., Jans, D. A., Sobolev, A. S. Insulin-mediated intracellular targeting enhances the photodynamic activity of chlorin e6. Cancer Res. 55:1014-1019 1995.

Bachor, R., Shea, C. R., Gillies, R., Hasan, T. Photosensitized destruction of human bladder carcinoma cells treated with chlorin e6-conjugated microspheres. Proc Natl Acad Sci USA 88:1580-1584 1991.

Cavanaugh, P.G. and Nicolson, G. L. Lung derived growth factor that stimulates the growth of lung-metastasizing tumor cells: Identification as transferrin. Journal of Cellular Biochemistry 47:261-271, 1991.

Cavanaugh, P. G., and Nicolson, G. L. The selection of a metastatic rat mammary adenocarcinoma cell line from a low metastatic parental population by an in vitro process based on cellular ability to proliferate in response to transferrin. Journal of Cellular Physiology. 174: 48-57 1998.

Cavanaugh, P. G., Jia, L., and Nicolson, G.L. Transferrin receptor overexpression enhances transferrin responsiveness and the metastatic growth of a rat mammary adenocarcinoma cell line. Breast Cancer Research and Treatment 56:203-217, 1999.

Conrad, M. E., and Umbreit, J. N. Iron absorption and transport-an update. Am J Hematol 64:287-298, 2000.

Del Govematore, M., Hamblin, M. R., Shea, C. R., Rizvi, I., Molpus, K. G., Tanabe, K. K., and Hasan, T. Experimental photoimmunotherapy of hepatic metastases of colorectal cancer with a 17.1 Å chlorin(e6) immunoconjugate. Cancer Res. 60:4200-4205, 2000.

Donald, P. J., Cardiff, R. D., He, D. E., Kendall, K. Monoclonal antibody-porphyrin conjugate for head and neck cancer: the possible magic bullet. Otolaryngol Head Neck Surg 105:781-787 1991.

Elliot, R. L., Stjernholm, R., and Elliot, M. C. Preliminary evaluation of platinum transferrin (MTPC-63) as a potential nontoxic treatment for breast cancer. Cancer Detect. Prev. 12: 469-480, 1988

Gatter, K. C., Brown, G., Trowbridge, I. S., Woolston, R. E., Mason, D. Y. Transferrin receptors in human tissues: their distribution and possible clinical relevance. J Clin Pathol 36: 539-545. 1983.

Gijsens, A., De Witte, P. Targeting of chlorine E6 by EGF increasing its photodynamic activity in selective ways. Verh K Acad Geneeskd Belg 62:329-352, 2000.

Hamblin, M. R., and Newman, E. L. Photosensitizer targeting in photodynamic therapy. I. conjugates of haematoporphyrin with albumin and transferrin. J. Photochem Photobiol B. 26: 45-56, 1994.

Hudson, P. J. Recombinant antibody constructs in cancer therapy. Curr Opin Immunol 11:548-557, 1999.

Hsi, R. A., Rosenthal, D. I., Glatstein, E. Photodynamic therapy in the treatment of cancer: current state of the art. Drugs 57:725-734 1999.

Katsumi, T., Aizawa, K., Okunaka, T., Kuroiwa, Y., Li, Y., Saito, K., Konaka, C., and Kato, H. Photodynamic therapy using a diode laser with mono-L-aspartyl chorin e6 for implanted fibrosarcoma in mice. Jpn J Cancer Res. 85:1165-1170, 1994.

Kemp, J. D., Smith, K. M., Mayer, J. M., Gomez, F., Thorson, J. A., and Naumann, P. W. Effects of anti-transferrin receptor antibodies on the growth of neoplastic cells. Pathobiology 60:27-32, 1992.

Kemp, J. D., Cardillo, T., Stewart, B. C., Kehrberg, S. E., Weiner, G., Hedlund, B., and Naumann, P. W. Inhibition of lymphoma growth in vivo by combined treatment with hydroxyethyl starch deferoxamine conjugate and IgG monoclonal antibodies against the transferrin receptor. Cancer Res. 55: 3817-3824, 1995.

Kovar, J., Naumann, P. W., Stewart, B. C., and Kemp, J. D. Differing sensitivity of non-hematopoetic human tumors to synergistic anti-transferrin receptor monoclonal antibodies and deferoxamine in vitro. Pathobiology 63: 65-70, 1995.

Niitsu, Y., Kohgo, Y., Nishisato, T., Kondo, H., Kato, J., Urushizaki, Y., and Urushizaki, I. Transferrin receptors in human cancerous tissues. Tohoku J Exp Med 153:239-243, 1987.

Ponka, P., Beaumont, C., and Richardson, D. R. Function and regulation of transferrin and ferritin. Seminars in Hematology 35: 35-54, 1998.

Ponka, P., and Lok, C. N. The transferrin receptor: role in health and disease. Int J Biochem Cell Biol 31: 1111-1137, 1999. Rossi, M. C. and Zetter, B. R. Selective stimulation of prostatic carcinoma cell proliferation by transferrin. Proc. Natl. Acad. Sci. USA, 89: 6197-6201, 1992.

Scott, A. M., Welt, S. Antibody-based immunological therapies. Curr Opin Immunol 9:717-722, 1997.

Seymour, G. J., Walsh, M. D., Lavin, M. F., Strutton, G., and Gardiner, R. A. Transferrin receptor expression by human bladder transitional cell carcinomas. Urol. Res. 15: 341-344, 1987.

Singh, M. Transferrin as a targeting ligand for liposomes and anticancer drugs. Curr Pharm Des 5:443-451, 1999.

Stackpole C W, Kalbag S S, Groszek L: Acquisition of in vitro growth autonomy during B16 melanoma malignant progression is associated with autocrine stimulation by transferrin and fibronectin. In Vitro Cell Dev Biol 31: 244-251, 1995

Testa, U., Pelosi, E., and Peschle, C. The transferrin receptor. Crit. Rev. Oncog., 4:241-276, 1993.

Van Muijen, G. N., Ruiter, D. J., Hoefakker, S. and Johnson J. P. Monoclonal antibody PAL-M1 recognizes the transferrin receptor and is a progression marker for metastasis. J. Invest. Dermatol. 95: 65-69, 1990.

Van Muijen, G. N. P., Jansen, K. F. J., Cornelissen, I. M. H. A., Smeets, D. F. C. M., Beck, J. L. M., and Ruiter, D. J. Establishment and characterization of a human melanoma cell line (MV3) which is highly metastatic in nude mice. Int. J. Cancer 48:85-91, 1991.

Wrba, F., Ritzinger, E., Reiner, A., and Holzner, J. H. Transferrin receptor (TriR) expression in breast carcinoma and its possible relationship to prognosis. An immunohistochemical study. Virchows Arch. 41: 69-73, 1986.

Yoda, J., Yamanaka, N., Saito, T., Samukawa, T., Tamura, S., and Kawaguchi, T. Characterization of cell lines from metastatic maxillary cancer. Journal of the Oto-Rhino-Laryngological Society of Japan 97: 419-429, 1994.

### BRIEF SUMMARY OF THE INVENTION

Human iron-saturated transferrin was bound to quaternary-amino ethyl (QAE) sephadex in a buffer of 25 mM sodium phosphate, pH 7.2, containing 2 mM of the detergent CHAPS (PB/CHAPS buffer). The gel was washed free of unbound transferrin and was reacted directly with 1-Ethyl-3-[3-dimethylaminopropyl] carbodiimide hydrochloride (EDC) and the porphyrin chlorin e6, in the same buffer. Or, chorin e6 was reacted with EDC in a separate vessel, in the PB/CHAPS buffer, and unreacted chorin e6 removed from the mixture by adsorption to QAE-sephadex, all in PB/CHAPS. This latter soluble EDC-modified chlorin e6 was added to the immobilized transferrin to produce the immobilized conjugate. In either case, the transferrin was conjugated while bound to the gel and was washed free of un-reacted soluble conjugation components. The conjugate was then released from the gel by treatment with PB/CHAPS containing 0.5 M NaCl. The conjugate was dialyzed against PB for further use.

The conjugate was first shown to retain transferrin's growth promoting activity on the rat MTLn3 tumor line, in a low serum growth assay. The conjugate was then tested for its ability to compete with FITC-transferrin for binding to the transferrin receptor, using a western blot-mediated ligand binding assay. The conjugate was seen to possess an altered migratory pattern when analyzed by native gel electrophoresis. Finally, the conjugate was seen to kill tissue cultured tumor cells in a light-exposure dependent fashion. This killing effect was not evident in the absence of light or when excess un-conjugated transferrin was present, indicating a specific effect. Chlorin e6-transferrin prepared in this manner retains biological activity and is a candidate for use as a photodynamic therapy treatment of cancer and other disorders.

The invention presents a novel method for the conjugation of a porphyrin to a protein, in particular, the conjugation of chlorin e6 to transferrin. This results in the formation of a relatively tumor-specific ligand which possesses cell killing activity when activated by photodynamic therapy. Although the use of transferrin as an anti-tumor photodynamic therapy agent has been discussed by others, the use of chlorin e6, the use of this conjugation technique, and an illustration of putative effect as presented here is not evident in the scientific or patent literature.

### BRIEF DESCRIPTION OF THE DRAWINGS

FIG. 1. A: Schematic of chorin e6. B: Schematic of the reaction of chlorin e6, EDC, and transferrin.

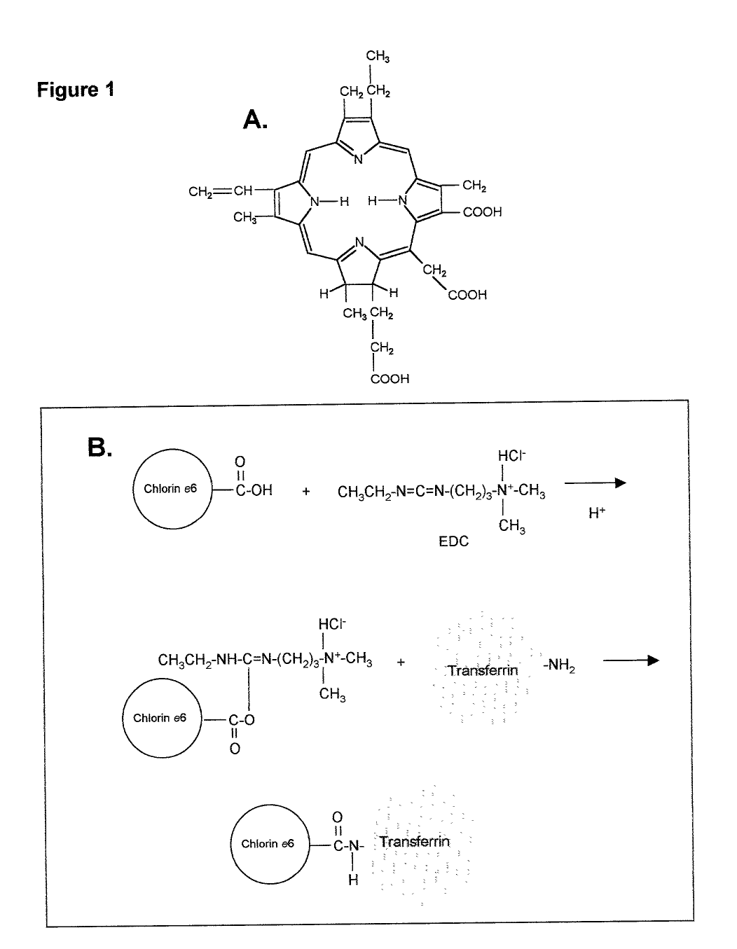

FIG. 2. Effect of chorin e6 on the growth of Rat MTLn3 mammary adenocarcinoma cells. Cells were plated at 2,000 cells/well in 96 well plates in ∝MEM containing 5% FBS. One day after plating, media was changed to ∝MEM containing 0.3% FBS. Increasing levels of human holo-Tf (Native Tf) or human Ce6-Tf (both in PBS) were added to respective wells, in the amount indicated. Four days later, cells were quantitated using a crystal violet stain assay, where A590 correlates with cell number. A: an image of the crystal violet stained plate used in the assay is shown. B: A plot of the absorbances from A. Cell number and is seen to rise as the cells are exposed to increasing levels of native human Tf. A similar, albeit slightly lower rise was seen with Ce6-Tf, indicating intact biological activity in the latter.

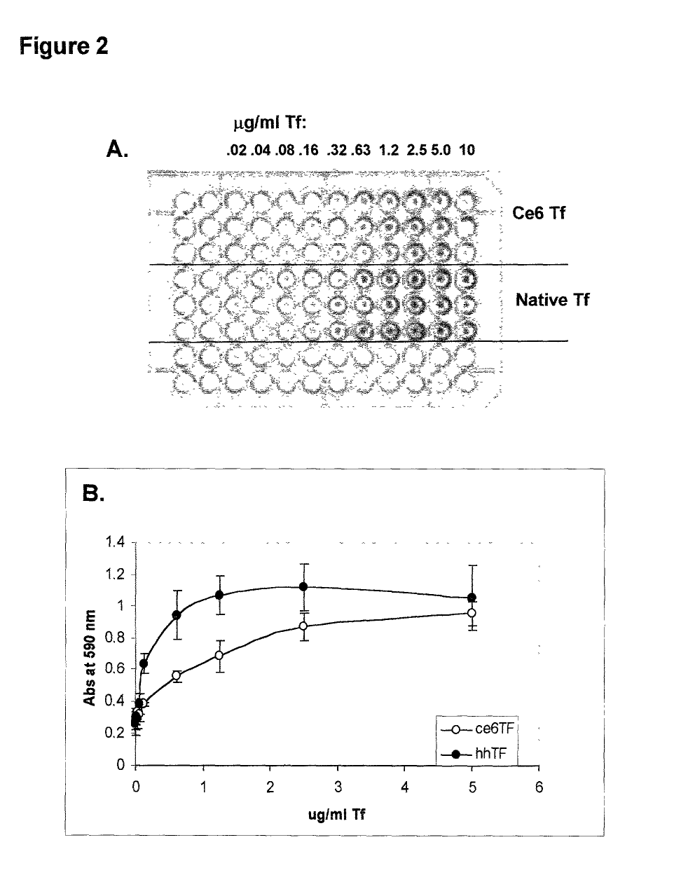

FIG. 3. Native gel electrophoretic analysis of Ce6-Tf. 10 μg quantities of all proteins listed were treated, loaded, and run out using the native gel system. The gel was fixed and stained with Coomassie blue. The results indicate a greater mobility of chlorin e6-20 transferrin (lanes 5 and 6) when compared to native transferrin (lane 4).

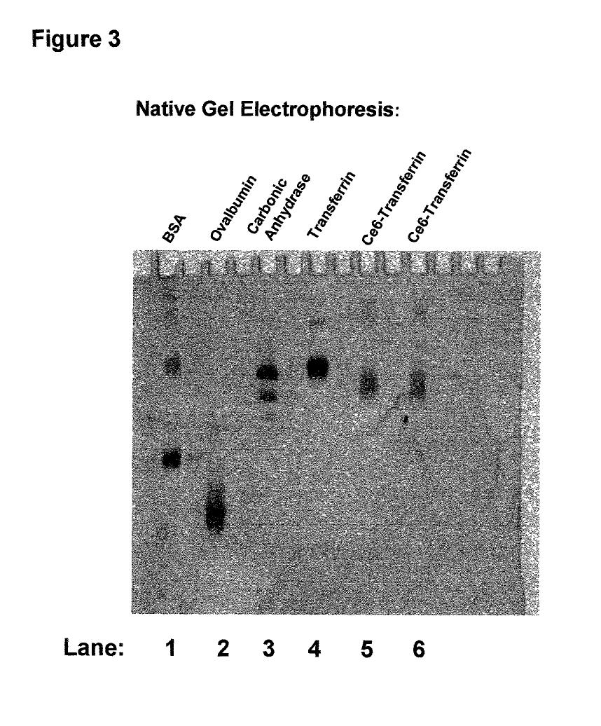

FIG. 4. Competition of FITC-Tf binding to cell surfaces by Ce6-Tf. Transferrin solvent, human Ce6-Tf, or native human Tf were added to Rat MTLn3 mammary adenocarcinoma cell monolayers equilibrated to 4° C. The final concentration of both transferrins was 1 mg/ml. FITC- human Tf was then added to all wells at 100 μg/ml. After a 2 h incubation, cells were washed, lysed, electrophoresed, blotted, and examined for FITC content by incubation with anti-FITC and an HRP-conjugated second antibody, followed by ECL. A strong band at 70,000 Kd was seen from cell lysates which received FITC-Tf only (lanes 4 and 5), indicating FITC-Tf binding to the cells. Both Ce6-Tf (lanes 6 and ) and native Tf (lane 8) competed out the FITC-Tf as indicated by the absence of any FITC signal in lysates from cells treated with either. Lanes 1-3 were loaded with known amounts of pure FITC-Tf, for standardization. An image of the ECL X-ray film is shown. The results indicate functional binding of Ce6-Tf to the transferrin receptor.

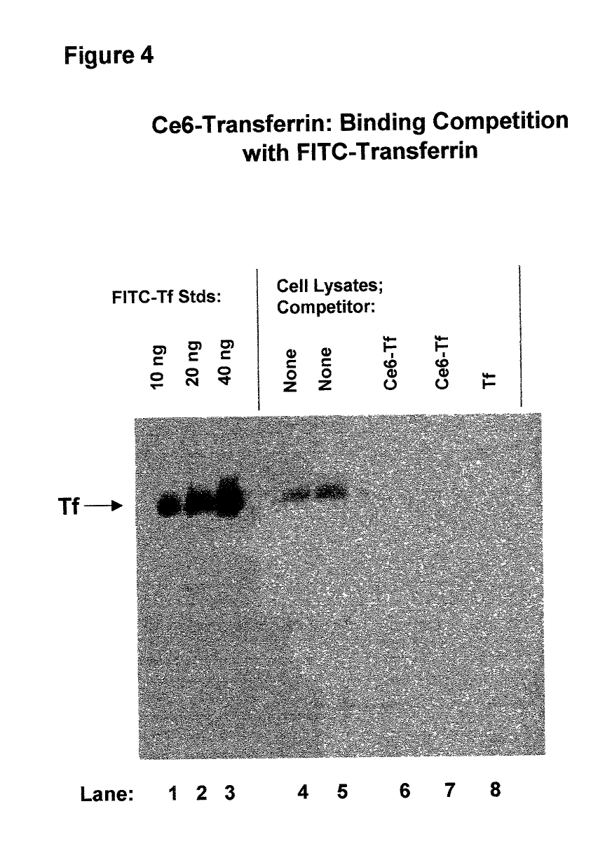

FIG. 5. Light-dependent killing of rat MTLn3 mammary adenocarcinoma cells by Ce6-Tf. This was a continuous exposure, serum-free assay performed using protocol A described in the cell killing section. Cells were plated in 24 well plates and grown to confluency in ∝MEM containing 5% v/v FBS. On day one, media was changed to ∝MEM only and increasing levels of Ce6-Tf were added to test wells to a final concentration from 1.25 to 5.0 μg/ml. Native Tf was added to control wells at 5.0 μg/ml. On days 2, 3, and 4, cells were exposed to light from an X-ray film box for 15 min. Media and all Tf was changed each day. On day five, all cells were quantitated using the crystal violet stain assay. Images of the stained plates are shown in A. Stained cell numbers were evaluated using a Bio-Rad Multi-imager. The results of image analysis are shown in B, where ODU/mm2 correlates with cell number. Results indicate a light-dependent killing as plates kept in the dark during the process displayed no loss of cell numbers.

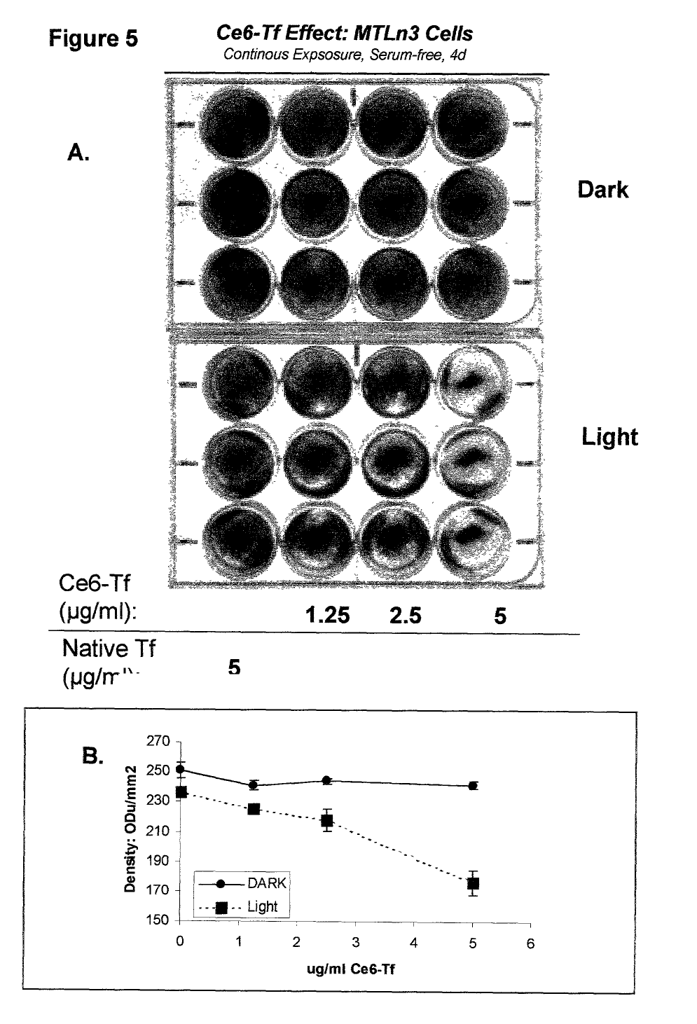

FIG. 6. Light-dependent killing of MTLn3 and NRK cells by Ce6-Tf. This was a one-day exposure, serum-containing assay performed using protocol B described in the cell killing section. Cells were plated in 24 well plates and grown to confluency. On day one, media was changed and increasing levels of Ce6-Tf were added to test wells to a final concentration from 7.5 to 30 ug/ml. Native Tf was added to control wells at 30 ug/ml. On day 2, media was changed to that without added Ce6-Tf or Tf. On days 2, 3, and 4, cells were exposed to light from an X-ray film box for 15 min. Media was changed each day. On day five, all cells were fixed, stained, and quantitated using the crystal violet stain assay. Stained cell numbers were evaluated using a Bio-Rad Multi-imager. Images of the stained plates are shown in A and B. The results of image analysis are shown in C and D, where ODU/mm2 correlates with cell number. Results indicate a light-dependent killing as plates maintained in the dark during the process displayed no loss of cell numbers. The MTLn3 cell line was more susceptible to the effects of the Ce6-Tf as it showed a decrease in cell numbers at the 15 ug/ml dose whereas the normal NRK line did not.

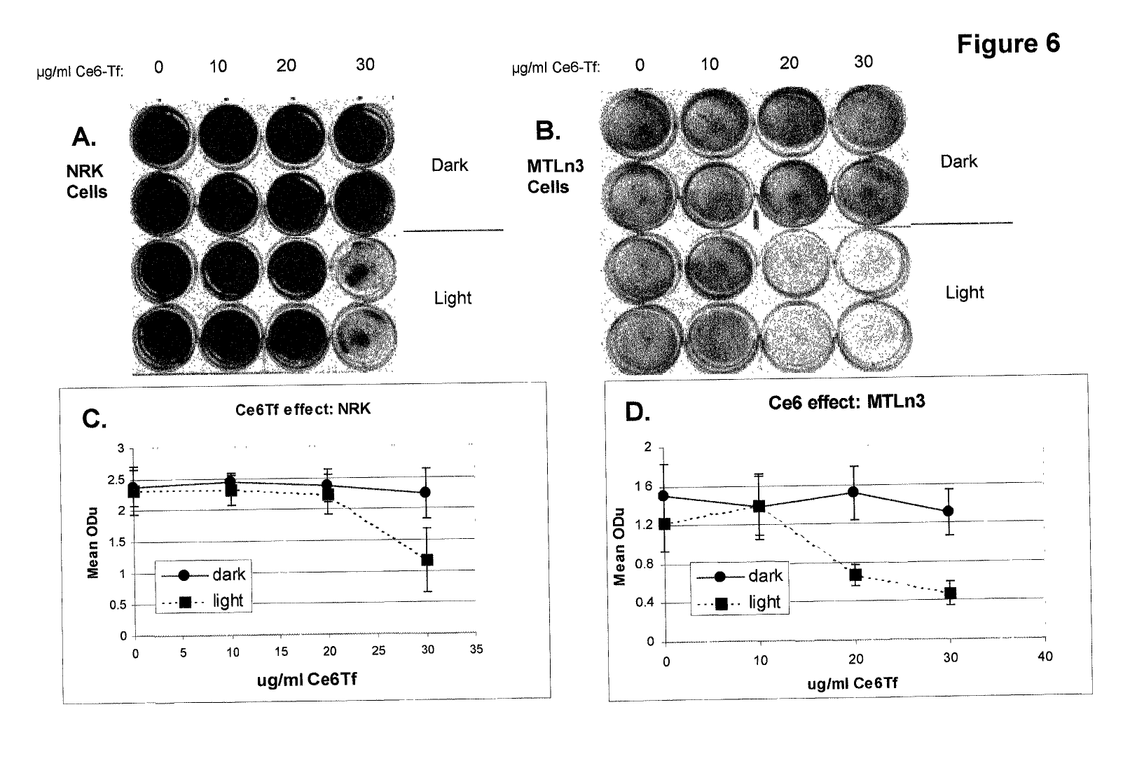

FIG. 7. Light-dependent killing of Human MCF7 breast cancer cells by Ce6-Tf This was a one-day exposure, serum-containing assay performed using protocol B described in the cell killing section. Cells were plated in 24 well plates and grown to confluency. On day one, media was changed and increasing levels of Ce6-Tf were added to test wells to a final concentration from 7.5 to 30 ug/ml. Native Tf was added to control wells at 30 ug/ml. On day 2, media in all wells was changed to that without added Ce6-Tf or Tf On days 2, 3, and 4, cells were exposed to light from an X-ray film box for 15 min. Media was changed each day. On day five, all cells were fixed, stained, and quantitated using the crystal violet stain assay. Stained cell numbers were evaluated using a Bio-Rad Multi-imager. Images of the stained plates are shown in A. The results of image analysis are shown in B, where ODU/mm2 correlates with cell number. Results indicate a light-dependent killing as plates kept in the dark during the process displayed no loss of cell numbers. As with the rat lines studied previously, this human line was also shown to be susceptible to a combination of Ce6-Tf and light.

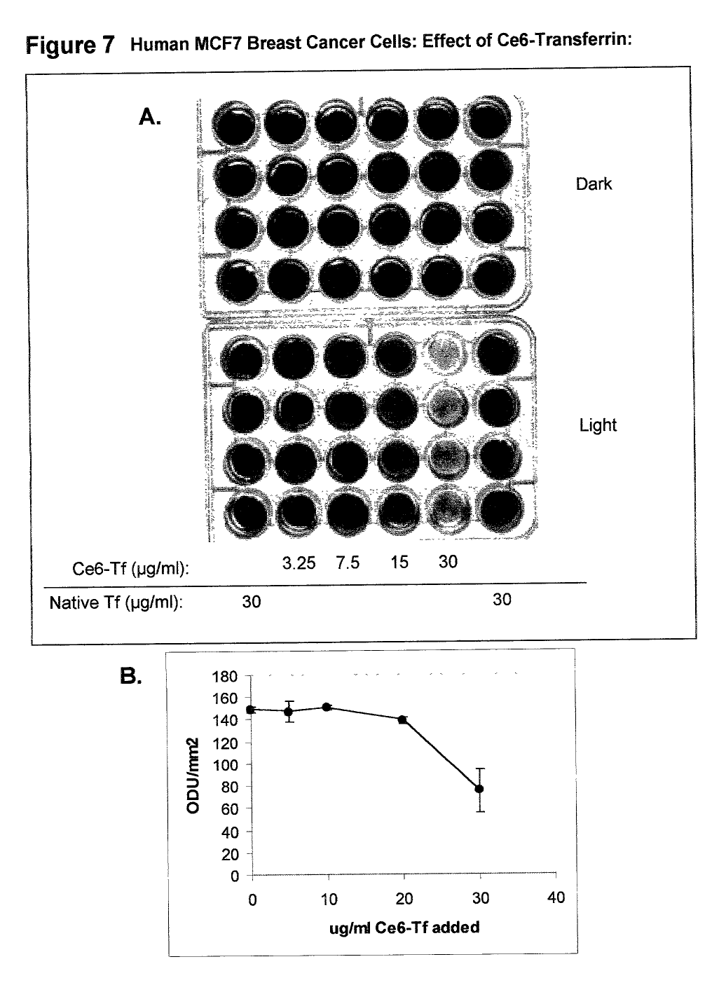

FIG. 8. A: Effect of Ce6 alone on the viability of Rat MTLn3 cells. Cells were tested as per method B outlined in the cell killing procedure description. Confluent cells in ∝MEM containing 5% FBS were exposed to the indicated concentrations of Ce6, Ce6-Tf, or Tf alone. One day later, media was changed to that without added Ce6-Tf or Tf, and all cells were exposed to light for 15 min. This was repeated on days two and three. Cells were then fixed and stained with Coomassie blue. An image of the stained wells is shown. The results indicate that Ce6 alone had no cell killing effect. B: Effect of excess Tf on the killing effect of Ce6-Tf Cells were set up similarly as above, except that treatments consisted of Ce6-Tf, or Ce6-Tf in conjunction with 500 or 1,000 μg/ml native Tf. Light exposure, media changes, and cell staining were carried out as in A. An image of the stained wells is shown. The results indicate that excess native Tf diminished the killing effect of Ce6-Tf, indicating that the latter acts through a Tf-specific process.

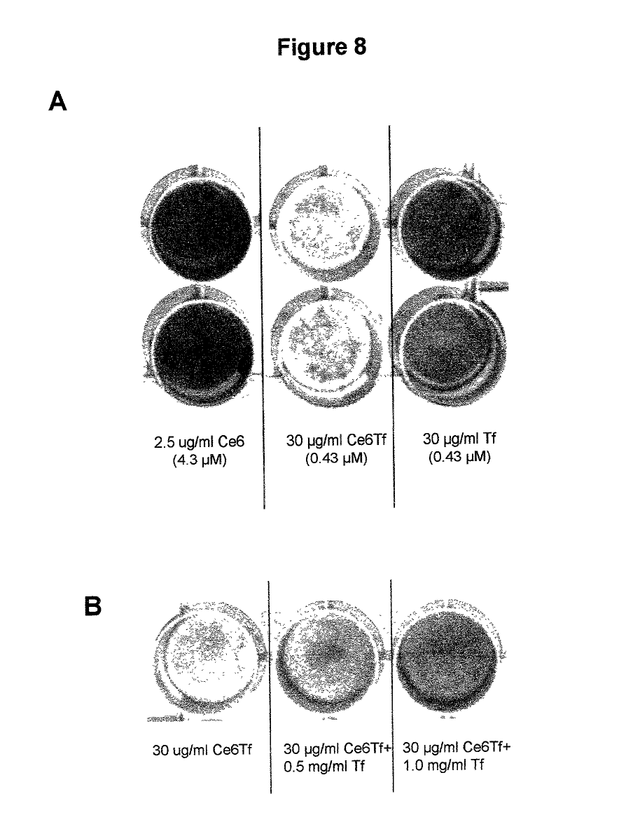

### DETAILED DESCRIPTION OF THE INVENTION

Synthesis of chorin e6-transferrin: QAE sephadex A-50 (Sigma Chemical) was hydrated fully in water at a ratio of 1:100 (gel:water; w:v). The suspension was centrifuged at 1,000 X g for 5 min and the gel pellet equilibrated in 50 volumes of phosphate buffer (PB; 20 mM Na2HPO4, pH adjusted to 7.4 with KH2PO4). The gel was re-centrifuged and equilibrated in 10 volumes of phosphate buffer containing 2 mM CHAPS (3-[(3-cholidamidopropyl) dimethylammonio]-1-propane-sulfonate; buffer=PB/CHAPS). This was centrifuged at 1,000 X g for 5 min and the gel maintained in a minimal volume of PB/CHAPS. Iron-saturated human transferrin (Sigma Chemical) was dissolved in PB/CHAPS to a concentration of 10 mg/ml. To 2 ml of Tf solution was added 0.5 ml of equilibrated QAE-sephadex slurry. This was mixed slowly by rocking for 30 min. The gel was washed three times by suspension in and centrifugation from 25 ml PB/CHAPS. To ensure saturation of the gel, the transferrin binding process was repeated. To make the conjugate, to 0.5 ml of QAE-sephadex-Tf was added 0.5 ml of a 2 mg/ml chlorin e6 solution (Porphyrin products; Logan, Utah), dissolved in PB/CHAPS. To this was added 150 uL of 10 mg/ml EDC (1-Ethyl-3-[3-dimethylaminopropyl] carbodiimide hydrochloride; Pierce Chemical), dissolved in water. This mixture was rocked for 20 min at 25° C. The mixture was centrifuged at 1,000 X g for 5 min and the supernatant removed. To ensure complete conjugation, an additional 0.5 ml of chlorin e6 and 150 uL of EDC were added to the gel. The gel mixture was rocked again at 25° C. for 25 min and the gel was washed four times by repeated suspension in and centrifugation (1,000 X g for 5 min) from 25 ml of PB/CHAPS. To elute the conjugated Tf, the gel was suspended in Iml PB/CHAPS containing 0.5 M NaCl. This was rocked for 20 min at 25° C., centrifuged at 1,000 X g for 5 min, and the supernatant collected. The elution step was repeated on the gel pellet and the supernatants pooled. The pooled chlorin e6-transferrin was dialyzed overnight at 4° C. against 4 L of PB containing 0.15 M NaCl.

Additional procedure for the removal offree chlorin e6: The pooled chlorin e6-transferrin (Ce6Tf) is dialyzed at 4° C. against 25 mM sodium acetate, pH 4.8. To eliminate remaining un-conjugated chlorin e6, the dialysate is combined with 2 ml of packed SP-sepharose, previously equilibrated in the same buffer. This is mixed for 30 min at 25° C. and the gel is washed three times by centrifugation from and re-suspension in 20 ml equilibration buffer. The bound chlorin e6-transferrin is released from the gel with 25 mM sodium phosphate, pH 7.2 ,containing 1.0 M NaCl. The released material is combined with 1/100 volume of 1% (w/v) ferric ammonium citrate, and dialyzed against 25 mM NaH2PO4, pH 7.2. With this procedure, transferrin possesses a net positive charge at a pH of 4.8, whereas un-modified (free) chlorin e6 retains a net negative charge. Therefore,the transferrin will bind to a negatively charged matrix, and the free chlorin e6 will not. This allows for the removal of free chlorin e6 via the washing procedure.

Additional procedure for the preliminary preparation ofEDC-chorin e6: Chlorin e6 is dissolved at 1 mg/ml in 25 mM sodium phosphate, pH 7.2 containing 2 mM CHAPS. One tenth volume of 10 mg/ml EDC (in water) is added and allowed to react with the chlorin e6 at room temperature for 20 minutes. This is combined with an equal volume of a 50% (vol/vol) slurry of QAE-sepharose suspended in and equilibrated in 25 mM sodium phosphate, pH 7.5, containing 2 mM CHAPS. The gel-reacted chlorin e6 mixture is allowed to react at room temperature for 20 minutes. The mixture is centrifuged at 1000 X g for 10 minutes and the modified chlorin e6 in the resulting supernatant is removed and added to QAE-sepharose immobilized transferrin for production of the conjugate as stated above. With this procedure, non EDC-reacted chlorin e6 will retain a net negative charge and will bind to the QAE-sepharose. Chlorin e6 which has reacted with the EDC at two or more carboxyls will possess a net positive charge and will not bind to the QAE-sepharose. Therefore, only modified chlorin e6 will be added to the protein and non-specific adherence of chlorin e6 to the QAE-sepharose-transferrin will be avoided.

Native gel analysis of chorin e6-transferrin: The acrylamide gel solution consisted of 0.37 M Tris, 0.17 M HCl, 9.75% w/v acrylamide, 0.25% w/v Bis-acrylamide, 2 mM CHAPS, 0.01% v/v TENED and 0.025% w/v ammonium persulfate. This was poured into a 15 ×15 ×0.1 cm chamber and polymerized. Samples were treated by addition of one third volume of 1.48 M Tris, 0.68 M HCl, 8 mM CHAPS, 0.01% w/v bromophenol blue, and 20% v/v glycerol. Samples were loaded onto the acrylamide gel and the gel was placed into an electrophoresis chamber containing an anolyte of 20.16 M Tris, 0.01 N HCl. A catholyte of 0.02 M glycine and 0.01 N KOH was overlaid onto the gel and the samples were electrophoresed at 40 mA constant current until the dye front was 1 cm from the bottom of the gel. The gel was fixed in 40 methanol, 10% acetic acid and was stained in fixative containing 0.2% Coomassie blue R250. The gel was destained with fixative.

Competition binding: This measures the ability of a material to inhibit the binding of FITC-transferrin to cell surfaces. FITC-Tf bound to the cells is detected by Western blotting of cell lysates and specific antibody-based detection of FITC in those. Rat MTLn3 mammary adenocarcinoma cells were grown to confluence in 12 well plates using media consisting of ocMM containing 5% v/v fetal bovine serun (FBS). Media was changed to ∝MEM only for 2 h an then again for overnight. The cells were equilibrated to 4° C., wells were drained and 1 ml of a binding buffer consisting of ∝MEM containing 25 mM HEPES (pH 7.5) and 3 mg/ml liquid gelatin was added to all wells. Ce6-TF to be tested was added to respective wells to a concentration of 1 mg/ml. Native human transferrin, as a known control inhibitor, was added to positive control wells to a concentration of 1 mg/ml. Negative control wells received transferrin buffer only. FITC-Tf was added to control and test wells to a concentration of 100 ug/ml. Cells were incubated at 4° C. for 2 h. All wells were washed 4 times with 2 ml PBS and cells were lysed with 0.5 ml PBS containing 2% Triton X-100, 0.1 U/ml aprotinin, and 100 μg/ml PMSF. Lysate protein was determined using the BCA assay (Pierce Chemical). Equal protein amounts of cell lysates were treated with SDS-PAGE treatment solution, were separated by SDS-PAGE, and blotted onto nitrocellulose. The blot was blocked and FITC-TF was detected by treatment with rabbit anti-FITC then with anti-rabbit IG-HRP followed by ECL using an HRP substrate.

Growth assays: Rat MTLn3 mammary adenocarcinoma cells were plated at 2,000 cells/well in 96 well plates in ∝MEM containing 5% FBS. One day after plating, media was changed to ∝MEM containing 0.3% FBS. Increasing levels of human holo-Tf or human Ce6-Tf (both in PBS) were added to respective wells. Four days later, cells were quantitated using a crystal violet stain assay.

Cell killing assays: A: Serum-free media assays. These were performed to initially assess the effect of Ce6-Tf and to verify its light-dependent killing. Target cells were grown to confluence in 24 well plates. On the day of the assay, media in all wells was replaced with 1 ml of fresh serum-free media and increasing levels of Ce6-Tf added to test wells. Native human holo-Tf was added, at the highest Ce6-Tf dose, to control wells.

B: Serum-containing, one day exposure assays. For these, serum was maintained, to emulate in vivo conditions where excess endogenous normal transferrin would be present. In addition, the Ce6-Tf exposure was limited to I day to emulate a one time Ce6-Tf injection. Target cells were grown to confluence in 24 well plates. On the day of the assay, media in all wells was replaced with 1 ml of fresh serum-containing media and increasing levels of Ce6-Tf added to test wells. Native human holo-Tf was added, at the highest Ce6-Tf dose, to control wells. One day after Tf addition, media in all plates was changed to normal culture media (without Ce6Tf).

With both assay methods, two plates for each line to be tested were plated and treated identically. One day after Ce6-Tf addition, test plates were exposed to the light from an X-ray film box for 15 min.: the box was placed horizontally and the culture plates placed directly on the cover glass. The parallel plate from a given line was kept in the dark. The light treatment was repeated for 3 days. Media was changed (with [A] or without [B] added Ce6-Tf ) each day, to maintain cell viability. On the fourth day, the cells were quantitated using a crystal violet stain assay: wells were drained and washed 4 times with 2 ml PBS; cells were fixed with 1 ml 5% v/v glutaraldehyde (in PBS) at 25° C. for 20 min.; wells were washed 4 times with 2 ml distilled water and stained with 1 ml of a 1:1 (v:v) mixture of 0.2% (w/v) crystal violet and 100 mM CAPS (pH 9.0). Wells were drained and washed 4 times with 2 ml distilled water. After drying, cell density was determined using a Bio-Rad Multiimager, where ODU/mm2 correlates with cell number.

Transferrin competition of cell killing: These were performed to ensure that Ce6-Tf's cell killing effect was due to the function of the transferrin ligand: that the light-induced killing effect could be neutralized with excess native Tf Method B. from above was used. Confluent cultures of MTLn3 cells in 24 well plates were treated with 30 ug/ml of Ce6Tf. At the time of Ce6Tf addition, certain wells also received human holo-transferrin so that the final concentration was 0.5 or 1.0 mg/ml. One day later, media was changed to normal culture media. Light-induced killing assays were continued and cells quantitated as stated above in method B.

Effect of Ce6 alone: To determine if Ce6 alone, if added in appropriate concentrations, would induce cell death. Gel filtration analysis indicated no significant change in Tf's molecular weight after Ce6 conjugation (data not shown). It was assumed from this that less than 10 molecules of Ce6 were conjugated to each Tf protein. Ce6Tf was very active in causing light-induced cell death when initially present at 0.43 uM ( 30 ug/ml), so free Ce6 was added to cultures at 4.3 uM, a ten fold molar excess, to ensure that Ce6 was present in greater amounts than that encountered by cells when exposed to Ce6Tf So Ce6 was added to a final concentration of 2.5 μg/ml to confluent MTLn3 cells. Light-induced killing assays were conducted as stated in method B above.

1. A new method for the conjugation of chorin e6 to transferrin by first immobilizing transferrin to an anion exchange gel. as described in the summary of the invention: Synthesis of Chorin e6-transferrin. Said gel is, but is not limited to, quatemary aminoethyl-sepharose (hereafter referred to as QAE sepharose); all solid supports such as polystyrene, cellulose, etc., containing quaternary amine or positively charged functional groups can be used for the preparation of chorin e6-transferrin.

2. The claim of 1 where the immobilized transferrin is reacted with chlorin e6 in the presence of, but not limited to, 1-Ethyl-3-[3-dimethylaminopropyl] carbodiimide hydrochloride (hereafter referred to as EDC), in the presence of a detergent, and the synthesized conjugate released using high salt. The coupling agent is, but is not limited to EDC. Other commonly used compounds such as cyclohexyl-3(2-morpholinoethyl) carbodimide can serve the same function.

3. The claim of 1 and 2, where the presence of a detergent is required for optimum formation of and release of the conjugate from the gel. The detergent is, but not limited to 3-[(3-cholidamidopropyl)dimethylammonio]-1-propanesulfonate (hereafter referred to as CHAPS). Other detergents, such as octyl glucoside, Triton X-100, Tween20, etc. can serve the same function.

4. Preparation of transferrin-QAE sepharose. The claim of 1,2, and 3, wherein iron-free or iron saturated transferrin from any species is immobilized or bound to, but not limited to, quaternary aminoethyl-sepharose while in a solvent of, but not limited to, 20 mM phosphate buffer, pH 7.4 (20 mM Na2HPO4, adjusted to pH 7.4 with KH2PO4; hereafter referred to as PB ) containing a detergent of, but not limited to, CHAPS, at a concentration of, but not limited to, 2 mM (solvent hereafter referred to as PB/CHAPS); and the gel is washed free of unbound transferrin in like solvent, after binding occurs to saturation and completion.

5. Preparation of chlorin e6-transferrin-QAE sepharose. The claim of 1, 2, 3, and 4 where 4, but not limited to 4, volumes of chlorin e6 in, but not limited to, PB/CHAPS is added to 1, but not limited to 1, volume of washed transferrin-QAE sepharose, and to this is added 0.25, but not limited to 0.25, volumes of EDC in a solvent of, but not limited to, purified water; and this mixture is incubated for, but not limited to, 20 minutes, at, but not limited to, room temperature, all while mixing, or by the use or any methodology, to ensure a uniform reaction which proceeds to saturation and completion.

6. Preparation of chlorin e6-transferrin-QAE sepharose, alternate to aim 5. The claims of 1, 2, 3, and 4, where chlorin e6 at, but not limited to, 1 mg/ml, dissolved in, but not limited to, PB/CHAPS is combined with EDC at, but not limited to, 1 mg/ml (initially dissolved at, but not limited to, 10 mg/ml in, but not limited to, water), for, but not limited to, 20 minutes, at, but not limited to, room temperature, and subsequently exposed to an excess of QAE-sepharose in, but not limited to, PB/CHAPS for, but not limited to, 20 minutes, at, but not limited to, room temperature; wherein the desired modified chlorin e6 remains unbound to and is separated from the gel by, but not limited to, centrifugation. Where 4, but not limited to 4, volumes of this modified chlorin e6, is added to 1, but not limited to 1, volume of washed transferrin-QAE sepharose, and this mixture is incubated for, but not limited to, 20 minutes, at, but not limited to, room temperature, all while mixing, or by the use or any methodology, to ensure a uniform reaction which proceeds to saturation and completion.

7. The claim of 5 and 6 wherein the chlorin e6-transferrin-QAE-sepharose and other insoluble material is washed of free chorin e6, modified chlorin e6, and other soluble material by, but not limited to, repeated centrifugation from and re-suspension in a solvent of, but not limited to, the PB/CHAPS solvent of claim 5.

8. The claim of 7 wherein the formed chlorin e6-transferrin is released from QAE sepharose by exposure to, but not limited to, PB/CHAPS containing, but not limited to, 0.5 MNaCl.

10. The claim of 8 wherein the released chorin e6-transferrin is freed of the high salt buffer or placed in a new solvent system by, but not limited to, dialysis. The claim whereby other methodologies such as, but not limited to, gel filtration or ultrafiltration, are used to eliminate the salt from the chlorin e6-transferrin.

11. The claim of 10 whereby chlorin e6-transferrin is further purified by being placed in a low pH solvent of, but not limited to 25 mM sodium acetate, pH 4.8, and is reacted with a negatively charged matrix such as, but not limited to, sulfo-propyl sepharose, in a solvent of, but not limited to 25 mM sodium acetate, pH 4.8; whereby the chlorin e-transferrin binds to the matrix and any free, unmodified chlorin e6 does not.

12. The claim of 11 whereby chlorin e6-transferrin immobilized to sulfo-propyl sepharose is washed free of soluble material by, but not limited to, repeated centrifugation from and re-suspension in a solvent of, but not limited to, 25 mM sodium acetate, pH 4.8.

13. The claim of 10, 11, and 12 where the sulfo-propyl sepharose bound chlorin e6-transferrin is released by, but not limited to, PB/CHAPS containing, but not limited to, 1.0 M NaCl, and is placed in a new solvent by, but not limited to, dialysis.

14. The claim of 1, 10, and 13, where said transferrin-chlorin e6 conjugate is added to cells in culture. The cells are, but not limited to, tumor cells. The tumor cells are, but not limited to, breast cancers, melanoma, etc., and all other cells or tumor cells possessing substantial amount of finctional transferrin receptors or other factors causing transferrin binding to, association with, or internalization into the cells.

15. The claim of 14 where said cultured tumor cells or other cells associated with chorin e6-transferrin are damaged or destroyed by exposure to light.

16. The claim of 1, 10, and 13, where said chlorin e6-transferrin conjugate is delivered into tumor bearing humans or animals by, but not limited to, injection, or other methods such as, but not limited to, catheter, etc.

17. The claim of 16 where said chorin e6-transferrin-tumor cells residing in said humans or animals are damaged or destroyed by exposure to light, where said light is any light source capable of converting chlorin e6 to the toxic form, including, but not limited to, fluorescent, incandescent, and laser light.

18. The claim of 1, 10, 13, 16, and 17 where said transferrin is purified from, but not limited to, the blood, serum, or plasma of a cancer patient or animal, is then conjugated with chlorin e6, delivered into that patient or animal, and that patient's or animal's tumor(s) is irradiated by light.

19. The claim of 17 and 18 where tumor cells in the treated patient or animal are damaged or destroyed directly by the chorin e6-transferrin/light therapy, or indirectly from subsequent destruction of light-damaged tumor cells by other events such as, but not limited to, recognition and destruction of light-damaged tumor cells by the immune system, and the patient's or animal's prognosis is improved

20. The claim of 16, 17, 18, and 19 where circulating chorin e6-transferrin-tumor cells are destroyed by passage of the patient's blood through a light-irradiation instrument positioned outside the body.

21. The claims of 16, and 17, where transferrin-binding, associating, or internalizing cells other than tumor cells are selectively destroyed using these methods, in the treatment of other conditions or diseases.

22. The claims of 16 and 17 where treatment of cancer-bearing humans or animals by administration of chlorin e6-transferrin followed by light exposure is used as an adjunct treatment for cancer, or any other condition, alongside existing conventional or other treatments.

23. The claims of 16 and 17 wherein said treatment of humans or animals by administration of chlorin e6-transferrin followed by light exposure is repeated multiple times to eliminate disease or for other purposes.

24. The claims of 1, 14, 15, 16, and 17, wherein said treatment of cultured cells or humans or animals by administration of chlorin e6-transferrin followed by light exposure is used for any diagnostic or research purposes.

25. The claim of 1, 10, 13, 16, and 17, wherein said transferrin is likewise conjugated with chlorin e6 and utilized in any way, whether activated to the toxin form or not, or activated to the toxin form in any way, by any methodology.
